(19) United States
(12) Patent Application Publication
(10) Pub. No.: US 2025/0345036 A1
(43) Pub. Date: Nov. 13, 2025

(54) PORTABLE ULTRASOUND APPARATUS, SYSTEMS AND METHODS

(71) Applicant: DEEP BREATHE INC., London (CA)

(72) Inventors: Robert Thomas ARNTFIELD, London (CA); Alessandro GUBBINI, State College, PA (US); Blake VANBERLO, Lucan (CA); Delaney SMITH, Port Perry (CA); Marwan RAHMAN, Mississauga (CA); Amir ROSHANKAR, London (CA); Ross Thomas PRAGER, London (CA)

(21) Appl. No.: 18/933,868

(22) Filed: Oct. 31, 2024

Related U.S. Application Data
(60) Provisional application No. 63/644,752, filed on May 9, 2024.
(60) Provisional application No. 63/644,759, filed on May 9, 2024.

(51) Int. Cl. A61B 8/00; A61B 8/14; B06B 1/06

(52) U.S. Cl. CPC A61B 8/4488; A61B 8/14; A61B 8/4236; A61B 8/54; B06B 1/0625; B06B 2201/76

(57) ABSTRACT

Portable ultrasound apparatus, systems methods are disclosed for scanning anatomical regions of interest. The apparatus includes a housing adapted to be fixed in place over the region, a plurality of transducers positioned in a geometrical arrangement within the housing, and configured to emit ultrasound waves and receive echoes. The system also includes an ultrasound probe including the transducers, at least one processor, and a display. The at least one processor analyzes raw data received from the transducers to identify the likelihood of a condition affecting the anatomical region of interest and provides an indication when the likelihood exceeds a predetermined threshold.

### TECHNICAL FIELD

The disclosed exemplary embodiments relate to ultrasound systems, methods and apparatus and, in particular, to portable ultrasound systems, methods and apparatus.

### BACKGROUND

Ultrasound is a medical imaging technique that uses high-frequency sound waves to create images of structures within the body. Unlike other imaging modalities such as X-rays, ultrasound does not use ionizing radiation, making it a safer alternative for many diagnostic procedures.

Ultrasound devices operate by emitting sound waves at frequencies typically ranging from 1 to 25 MHz. These sound waves are directed into the body where they interact with tissues, organs, and fluids. The waves are reflected back to the ultrasound device where they are detected by a transducer. The transducer converts these reflected waves into electrical signals, which are then processed by the ultrasound machine to create visual images of the internal anatomy.

A-mode (or Amplitude Mode) ultrasound is the simplest form of ultrasound, primarily used to measure distances within the body. In A-mode, a single transducer sends and receives sound waves echoed or reflected from structures within the body. The amplitude of the peaks on the graph suggests the density of the tissues encountered by the sound wave and the position of the peaks suggests the depth at which those echoes were generated. However, the graph-like display can be difficult for non-experts to interpret, especially when comparing different signals or analyzing complex patterns.

M-mode (or Motion Mode) ultrasound is used to display the movement of organs or tissues along a single line, or ‘slice,’ over time. In this mode, an ultrasound machine continuously records the amplitude and depth of echoes from a moving organ, such as a breathing lung or beating heart. The M-mode display is a time-motion trace that shows how distances within the body change over time. M-mode provides a one-dimensional representation of the scanned area, which can make it difficult to visualize complex structures or movements. Moreover, as with A-mode imaging, the display requires expertise to interpret and may be difficult for non-experts to understand.

B-mode (or Brightness Mode, sometimes also known as 2D mode), is the most commonly used ultrasound mode for medical imaging. It provides a two-dimensional cross-sectional image of internal body structures. In B-mode ultrasound, the intensity of the echo is represented as a dot with varying degrees of brightness on a gray scale, and the generated image further includes a width dimension, thus producing an intuitive two-dimensional view. This mode is extensively used across many fields such as cardiology, obstetrics, thoracic and abdominal imaging to assess the structure and function of organs, detect lesions, and guide interventions. However, B-mode imaging depth may be limited by the frequency of the ultrasonic waves and the density of the tissue being imaged. Images can also be affected by artifacts caused by factors like patient movement, gas bubbles, or calcifications. Thus, although B-mode imaging is generally more visually intuitive, it still requires expertise to interpret and thus may be difficult for non-experts to understand.

### SUMMARY

In a broad aspect, there is provided an ultrasound apparatus for scanning an anatomical region of interest, the apparatus having: a housing adapted to be fixed in place over the anatomical region of interest; a plurality of transducers provided in the housing and positioned in a geometrical arrangement for scanning the anatomical region of interest while the housing is fixed in place over the anatomical region of interest, the plurality of transducers configured to emit a plurality of ultrasound waves and receive a plurality of echoes produced by said ultrasound waves.

Implementations may include one or more of the following features. Each of the plurality of transducers may have a single element with fixed focus. Each of the plurality of transducers may have one or more annular element. Each of the plurality of transducers may have a 1-D array of elements. Each of the plurality of transducers may have a 2-D array of elements.

The geometrical arrangement may cover an area larger than an anatomical region of interest. The anatomical region of interest may have an area between 25 cm2 and 400 cm2, or between 50 cm2 and 200 cm2, or preferably between 75 cm2 and 150 cm2. The anatomical region of interest may be selected from the group consisting of trachea, bronchi, bronchioles, alveoli, pleurae and pleural cavity.

The geometrical arrangement may optimize a coverage area of the plurality of transducers over the anatomical region of interest. The geometrical arrangement may be a 1-D geometrical arrangement or a 2-D geometrical arrangement. The geometrical arrangement may be a structured grid, such as a rectangular or hexagonal grid, which may have seven transducers, or an unstructured grid.

The housing may be flexible. A stick-to-skin adhesive may be provided on the housing for fixing the housing in place over the anatomical region of interest. A coupling gel may be provided on the housing for acoustically coupling the plurality of transducers.

A processor may be provided and configured to individually control the plurality of transducers. The processor may process the plurality of echoes using a machine learning model to identify a likelihood of a condition affecting the anatomical region of interest. In some cases, the processing includes categorizing the plurality of transducers into one or more included transducers and one or more excluded transducers, wherein the one or more included transducers are used in further processing to identify the likelihood of the condition.

The processor may be provided within the housing, or external to the housing.

A display may be provided on the housing and, when the likelihood exceeds a threshold percentage, the processor may transmit an indication to the display. The display may be an indicator light or a graphic display.

The transducer elements may be crystal, ceramic with piezoelectric properties, MEMS, or any combination thereof.

In another broad aspect, there is provided a portable ultrasound system for scanning an anatomical region of interest, the system having: an ultrasound probe including a plurality of transducers and adapted to be fixed in place over the anatomical region of interest; at least one processor; and a display.

Implementations may include one or more of the following features. The at least one processor may include a controller configured to generate one or more acoustic beams via the plurality of transducers. The controller may be further configured to sequence acquisition of raw data from the plurality of transducers. The at least one processor may be further configured to convert the raw data from the subset into data sets.

The at least one processor may be further configured to process the raw data or the data sets to identify an included subset of the plurality of transducers.

The at least one processor may be further configured to process the raw data or the data sets to identify an excluded subset of the plurality of transducers.

The at least one processor may be configured to adjust an angle of an acoustic beam associated with a selected transducer of the excluded subset of the plurality of transducers (or not of the included subset of the plurality of transducers), and, in response to determining that the selected transducer is to be included in the included subset, updating the included subset to include the selected transducer and/or updating the excluded subset to remove the selected transducer.

The processor may be configured to identify the included subset by analyzing the raw data or the data sets to identify a marker.

The processor may be configured to identify the excluded subset by analyzing the raw data or the data sets to identify an artifact.

The ultrasound probe may be adapted to be fixed in place over an anatomical region of interest.

The processor may be configured to identify an anatomical landmark in the data sets and, in response to identifying the anatomical landmark: determine an offset for the probe; and, display a user instruction to shift the ultrasound probe by the offset on the display.

The processor may be configured to analyze the data sets corresponding to the included subset to identify a likelihood of a condition affecting the anatomical region of interest. When the likelihood exceeds a predetermined threshold, the processor may be further configured to display an indication on the display.

The raw data may be A-mode data, B-mode data and/or M-mode data.

The controller may be configured to adjust one or more beam shape of the one or more acoustic beams.

The display may be an indicator light or a graphic display.

The condition may be a pathological condition affecting one or more of a patient's trachea, bronchi, bronchioles, alveoli, pleurae and pleural cavity.

The at least one processor may be provided within the housing or external to the housing.

In another broad aspect, there is provided a method for scanning an anatomical region of interest using a system or apparatus described herein.

In another broad aspect, there is provided a non-transitory computer readable medium, or a computer program product embodied in a computer readable medium, storing instructions that, when executed by at least one processor, cause the at least one processor to carry out a method as described herein, or implement a system or apparatus described herein.

### DESCRIPTION OF THE DRAWINGS

The patent or application file contains at least one drawing executed in color. Copies of this patent or patent application publication with color drawings will be provided by the Office upon request and payment of the necessary fee.

The drawings included herewith are for illustrating various examples of articles, methods, and systems of the present specification and are not intended to limit the scope of what is taught in any way. In the drawings:

FIG. 1 is a schematic drawing of an ultrasound system in accordance with at least some embodiments;

FIG. 2A is a schematic drawing of a single element transducer arrangement in accordance with at least some embodiments;

FIG. 2B is a schematic drawing of a multiple element transducer arrangement in accordance with at least some embodiments;

FIG. 2C is a schematic drawing of an array transducer arrangement in accordance with at least some embodiments;

FIG. 2D is a schematic drawing of an array-of-arrays transducer arrangement in accordance with at least some embodiments;

FIG. 2E is a schematic drawing of a digital transducer arrangement in accordance with at least some embodiments;

FIG. 3A is a schematic representation of a single element transducer;

FIG. 3B is a schematic representation of a multiple element transducer;

FIG. 3C is a schematic representation of a 1D array of single element transducers;

FIG. 3D is a schematic representation of a 1D phased array transducer;

FIG. 3E is a schematic representation of a 1D linear array transducer;

FIG. 3F is a schematic representation of a 2D array transducer;

FIG. 4 is a flow chart diagram of an example method of detecting a likelihood of a condition affecting an anatomical region of interest in accordance with at least some embodiments;

FIG. 5 is a flow chart diagram of another example method of detecting a likelihood of a condition affecting an anatomical region of interest in accordance with at least some embodiments;

FIG. 6 is an M-mode image showing a vertical slice through time taken from a B-mode video;

FIGS. 7A to 7F provide a schematic representation of the methods underlying each 3-second clip segment prediction;

FIGS. 8A to 8C provide a comparison of the lung sliding (FIG. 8A), absent lung sliding (FIG. 8B), and lung point (FIG. 8C) artifacts on B-Mode (i and ii) and M-Mode (iii and iv) ultrasound;

FIG. 9 is a still frame from a B-mode video with multiple pleural line fragments;

FIG. 10 is a still frame from a B-mode video where the pleural line ROI has been split into b=4 bins of equal width;

FIG. 11 is a visual representation of a method in accordance with at least some embodiments; and

FIG. 12 is a flow diagram of executable instructions for processing medical imagery of a lung.

### DETAILED DESCRIPTION

Ultrasound is a sophisticated diagnostic tool that, despite its apparent simplicity in operation, requires significant training and expertise to use effectively. The challenges and complexities involved make it difficult for laypersons to effectively operate ultrasound equipment or interpret the results.

For instance, ultrasound machines come with various settings that need to be adjusted according to the type of examination (e.g., depth, focus, gain, frequency of the probe). Each setting affects the quality and detail of the images produced, and incorrect adjustments can lead to poor image quality or misleading information.

Moreover, interpreting ultrasound images can be challenging for several reasons, including but not limited to:
Operator dependency: The quality of ultrasound images and their interpretation depends heavily on the skill and experience of the operator performing the examination. A proficient ultrasonographer is required to adequately position the probe (transducer) and acquire high-quality images, as well as accurately interpret them. Inadequate training or lack of familiarity with specific anatomy may lead to errors in image interpretation.
Anatomic complexity: Many anatomical structures can be challenging to visualize and interpret using ultrasound imaging due to their intricate nature, overlapping layers, varying depths, and small size. This difficulty is compounded by the fact that some organs or tissues may not have well-defined borders on ultrasound images (e.g., liver).
Image artifacts: Ultrasound imaging can be affected by various artifacts, which are inaccuracy of anatomical structures or image distortions caused by factors such as equipment limitations, operator error, patient movement, image processing or pre-processing, etc. Common artifact types include acoustic enhancement (appearance of deeper structures due to the lack of attenuation), mirror-image artifacts (duplication of structures in front and behind reflectors), and reverberation/artifacts caused by multiple reflections within tissues or interfaces between media with different acoustic properties.
Image processing: Human operators are unable to interpret raw ultrasound data. Accordingly, the raw data is converted to images. This image conversion process may lose or obscure relevant information contained in the raw data, or may introduce additional processing artifacts.
Inherent limitations: Ultrasound imaging has inherent limitations due to its reliance on the propagation of sound waves through biological tissues. Factors such as the variability in ultrasound beam penetration, scattering, and attenuation can affect image quality, making it more difficult to accurately interpret certain structures or pathologies within images.
Heterogeneity of anatomy: The variation in size, shape, density, and composition among patients' bodies means that standardizing ultrasound imaging techniques may be challenging. This diversity can impact image interpretation, as well-defined landmarks on one patient might not resemble those seen on another.
Limited depth penetration: High frequency sound waves (10-25 MHZ) such as those used in B-mode ultrasound imaging have excellent resolution but limited depth penetration into the body due to increasing attenuation with distance from the transducer. This limitation may hinder visualization of deep or dense structures like bone, gas-filled organs, and highly calcified tissues.

Given these complexities, existing ultrasound devices are not suitable for layperson use, or even use by clinicians who lack ultrasound training. The existing technology demands a combination of technical skills, detailed anatomical and physiological knowledge, and interpretative expertise to ensure safe and effective use.

The embodiments described herein enable self-directed lung ultrasound, allowing individuals who are not trained in ultrasound techniques to perform diagnostic assessments of their lungs or other anatomy. This is achieved through the use of an apparatus or probe device that can be placed at one or multiple locations over the anatomical region of interest. The simplicity and ease of use of this apparatus and system make it accessible to a wide range of users, including patients themselves or clinicians who may not have prior experience with ultrasound technology.

Once the apparatus or probe device is in place, ultrasound data is gathered and processed using machine learning models. These algorithms are designed to identify the likelihood of various conditions affecting the anatomical region of interest, such as lung disease or other pathology. By leveraging machine learning, the system can analyze complex patterns and relationships within the ultrasound data to provide accurate diagnoses without requiring expert interpretation.

The design and form factor of the apparatus or probe device play a role in enabling this self-directed diagnostic capability. The device is intuitive and easy to use, allowing users to gather the ultrasound data. This simplicity also makes it possible for individuals who are not trained in ultrasound techniques to perform the assessment without requiring extensive training or expertise.

By empowering patients and clinicians with the ability to perform self-directed lung ultrasound assessments, this system can improve diagnosis times, reduce costs, and enhance patient outcomes. Additionally, the machine learning-based analysis capabilities of the system can help identify patterns and trends that may not be apparent through traditional diagnostic methods, leading to more effective treatment and management strategies.

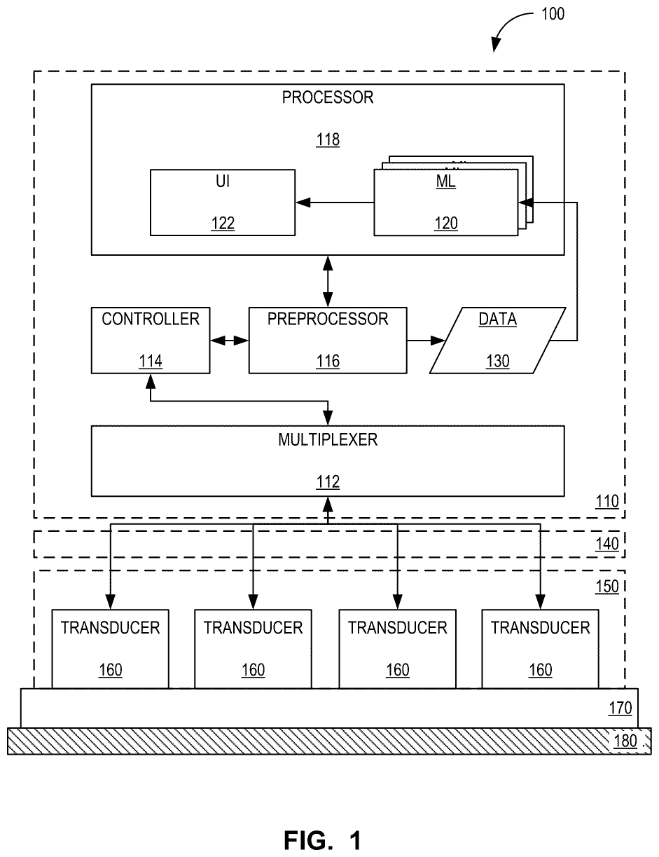

Referring now to FIG. 1, there is illustrated a schematic drawing of an ultrasound system in accordance with at least some embodiments. Ultrasound system 100 generally has one or more transducers 160 in a probe assembly 150 (also referred to as the ultrasound probe), which is supported by an electrical interconnection layer 140. The probe assembly 150 may be adapted to be fixed in place over the anatomical region of interest. There also may be an apparatus that comprises all or a portion of the ultrasound system 100 within a housing adapted to be fixed in place over the anatomical region of interest. For example, in some cases, there may an ultrasound apparatus that comprises the one or more transducers 160 in a housing adapted to be fixed in place over the anatomical region. In other cases, the ultrasound apparatus may comprise one or more other components of the system 100.

The transducers 160 are provided (e.g., in a housing or patch) and positioned in a geometrical arrangement for scanning the anatomical region of interest while the housing or patch is fixed in place over the anatomical region of interest, as described elsewhere herein. The housing or patch provides a stable platform for the transducers to emit and receive ultrasound waves. In some cases, the housing or patch may be fixed in place with a stick-to-skin adhesive. In some cases, the housing or patch may be fixed in place with straps, clips or a sleeve or similar means to securely attach or fit onto the body part being scanned, facilitating alignment of the transducers and reducing discomfort or irritation to the patient, particularly if the apparatus is worn for extended periods of time.

Additionally, an acoustic coupling gel may be provided on the housing or probe assembly 150 to acoustically couple the transducers 160 with the patient's body.

The probe assembly 150 or the housing or both may be flexible to accommodate the anatomy associated with the anatomical region of interest. For example, the probe assembly and/or housing may be made of a silicone-based material that is compliant with the body's natural curvature. This flexibility allows the probe assembly to conform to the shape of the body, ensuring optimal contact between the probe assembly and the tissue being imaged. Additionally, the flexible design enables the probe assembly to move freely, reducing the risk of damage or dislodgment during use. Likewise, the transducers 160 may be made of a flexible material, for similar reasons.

Generally, the geometrical arrangement has dimensions optimized to provide a coverage area of the transducers over the anatomical region of interest. In some cases, this may mean covering an area larger than an anatomical region of interest. For example, in at least some embodiments, the anatomical region of interest may be a trachea, bronchi, bronchioles, alveoli, pleurae, pleural cavity, or some portion thereof. In some example embodiments, the anatomical region of interest has an area between 25 cm2 and 400 cm2, and particularly between 50 cm2 and 200 cm2, and more particularly between 75 cm2 and 150 cm2.

The geometrical arrangement may be a one-dimensional arrangement (e.g., arranged in a line), or a two-dimensional arrangement.

In some cases, the geometrical arrangement is a structured grid, such as a hexagonal or regular or rectangular grid. In some cases, the geometrical arrangement may be unstructured, such as unstructured grid, with the positions of the transducers selected to optimize coverage over an anatomical region of interest. Unstructured in this context means that the transducers do not follow a predetermined or regular pattern. This can be useful for scanning complex anatomical regions or detecting subtle changes in tissue structure. In particular, the arrangement of the transducers can be tailored to specific scanning tasks or anatomical regions. The use of unstructured grids may also enable improved detection and characterization of subtle changes in tissue structure or motion. By having multiple transducers arranged in a non-linear pattern, the system can detect and track small movements or changes that might be missed by traditional linear arrays.

The transducers 160 are electrically coupled to a multiplexer 112 that is part of a processing assembly, which transmits and receives data from a controller 114 for transmitting ultrasound waves and receiving reflected ultrasound waves (i.e., echoes), respectively. The controller 114 may be implemented by a processor and thus may alternatively be referred to as a processor. This enables the transducers to generate one or more acoustic beams via the plurality of transducers. The transducers may produce raw data in A-mode, M-mode or B-mode. When the probe assembly is capable of acquiring three-dimensional data, the raw data may also be three-dimensional data.

Controller 114 can individually control each transducer 160 to provide fine-grained control, and to enable selection of active subsets of the transducers 160, which can be used to form included subsets of data (or excluded subsets).

Transducers 160 are provided in the probe assembly 150 according to a geometrical arrangement that is generally fixed in the X-Y plane (i.e., as viewed from the transmitting/receiving end) but can be flexible in the Z dimension to conform to the patient's body. This flexibility enhances acoustic coupling, and further allows for a more comfortable and secure fit, reducing the risk of movement or dislodgement during use. Each transducer 160 has at least one transducer element, which is the individual component made of piezoelectric materials—such as crystal or ceramic—or microelectromechanical systems (MEMS) that change shape or move in response to an applied electrical signal. This change in shape causes the transducer element to convert the electrical signal into a mechanical vibration, and vice versa—allowing for the transmission and reception of ultrasound waves.

Some transducers 160 have a plurality of transducer elements, which can be arranged in various configurations depending on the specific application. For example, in some cases multiple transducer elements may be arranged in an array to provide increased sensitivity or resolution. In other cases, individual transducer elements may be used to detect specific types of signals or vibrations. The use of multiple transducer elements can also allow for the detection of signals from different directions or angles, further increasing the versatility and effectiveness of the transducers.

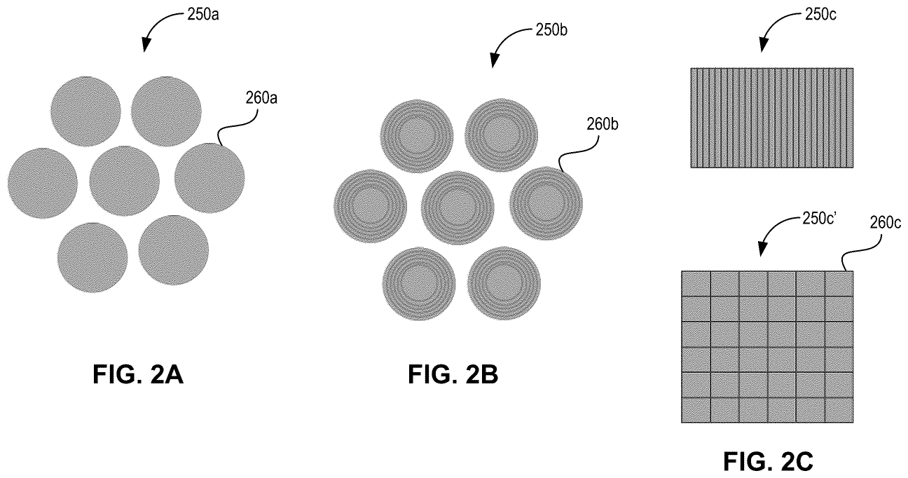

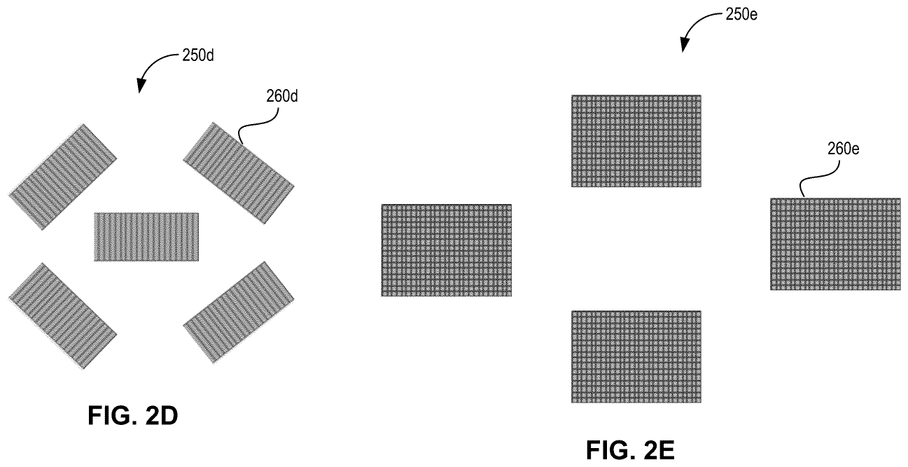

There may be one or more transducers 160 arranged in a variety of different arrangements as described elsewhere herein, particularly with reference to FIGS. 2A to 2E.

The controller is also configured to sequence acquisition of raw data from the transducers 160 via the multiplexer 112, and can also be used to operate a subset of the transducers 160. Generally, multiplexer 112 is provided when the number of transducer elements is greater than the number of available transmitting/receiving channels. If the number of transmitting/receiving channels is greater than the number of transducer elements, then the multiplexer 112 may be omitted.

The active (or “included”) subset of transducers may be selected during an initialization phase, and can be used to select only those transducers that are deemed to provide the most relevant data. For example, a preprocessor 116 or processor 118 may obtain raw data from controller 114 and analyze the data from each transducer to determine an included data set and, optionally, an excluded data set. The included data set consists of data that contains a marker or other information that is reflective of the anatomical region of interest. Conversely, the excluded data set consists of data that lacks the marker, or contains artifacts or other information that is not reflective of the anatomical region of interest (e.g., because the user has positioned the probe assembly 150 such that some or all of the transducers are not over the anatomical region of interest), or to avoid artifacts (e.g., due to poor acoustic coupling, or structures in the body).

For example, if the anatomical region of interest is a patient's lung, some transducers may be positioned such that the beam is reflected by a rib, which prevents acquisition of data from the tissue of interest (e.g., the lung or other structure inside the thoracic cavity). In such cases, the transducer in question may be excluded from the active subset of transducers and, corresponding, the data from the respective transducer is omitted from the included data set. Optionally, the data from the respective transducer may be assigned to an excluded data set. This can serve to reduce the processing load on processor 118, particularly when executing machine learning model 120, by eliminating the need to process data from a transducer that is unable to obtain relevant data at that moment. This artifact detection may be performed during the initialization phase by a machine learning model trained to identify such undesired artifacts.

In some embodiments, particularly if the probe assembly has beam steering ability, then, as part of the initialization phase, the processor may compute and adjust to a different acoustic beam angle and/or beam shape for a transducer that is not part of the active subset (or which is not part of the included subset). If the adjusted beam angle and/or beam shape causes the data to be relevant once again, the transducers may again be included in the active subset of transducers used for acquiring data, and its data in the included data set.

In some embodiments, the processor 118 may be further configured to, using a positioning machine learning model, identify an anatomical landmark in the included and/or excluded data sets and, in response to identifying the anatomical landmark: determine an offset for the probe assembly that is expected to produce improved data, and display a user instruction via the user interface 122 to shift the probe assembly by the offset. For example, the user instruction may be “move device down by 3 cm.”

In some embodiments, the user instruction may be provided in combination with, or as part of, a two-dimensional image such as a B-Mode anatomical image. The processor 118 may generate a B-Mode image and provide visual and/or textual indications. If the user instruction is provided in real-time, the indications may be updated in real-time to aid the user in positioning the apparatus or probe assembly. For example, the indications may be arrows, highlights and/or outlines to suggest a direction of repositioning and a desired target of the repositioning.

The initialization phase can also be repeated, if necessary, during scanning to select a new active subset of transducers whose data is in the included data set. For example, if the probe assembly 150 or the patient has been moved, the initialization phase may be repeated.

In some embodiments, transducers 160 that are not part of the active subset, i.e., whose data is not in the included data set, may be disabled briefly to reduce power use and also to reduce noise.

The controller 114 is in turn coupled to a preprocessor 116, such as a field programmable gate array (FPGA), application specific integrated circuit (ASIC) or other processor for obtaining and preprocessing (if applicable) the raw data from the controller, and for generating the signals used by the controller to transmit ultrasound waves of the desired frequency and phase. The controller, preprocessor and processor collectively may be referred to as at least one processor.

Data 130 output by preprocessor 116 is provided to a processor 118, which can execute one or more machine learning model 120 to identify the likelihood of a condition affecting the anatomical region of interest, or to guide positioning of the device, as described elsewhere herein, and provide an output via a user interface 122. The machine learning models may include, but are not limited to, neural networks, decision trees, and support vector machines. The models may be trained on a dataset that includes a wide range of inputs obtained directly from transducers, along with labeling that indicates the presence or absence of certain conditions.

The preprocessor 116 or the processor 118 may convert raw data received from controller 114 into data sets and, further, into included data sets. Once the initialization phase has been completed, the raw data may be filtered by the controller 114, preprocessor 116, and/or processor 118 to include only raw data from transducers that are part of the included data set.

When an included data set has been identified, generally the processor will attempt to identify the likelihood of the condition by executing the machine learning model 120 using the included subset as input. When the likelihood exceeds a predetermined threshold (e.g., threshold percentage), the processor is further configured to transmit an indication or display an indication on a display.

In at least some embodiments, the condition affecting the anatomical region of interest is a pathological condition affecting one or more of a patient's trachea, bronchi, bronchioles, alveoli, pleurae and pleural cavity. For example, this may include conditions such as chronic obstructive pulmonary disease (COPD), asthma, pneumonia, lung cancer, pleurisy, hemothorax, or pneumothorax.

Accordingly, aspects of the described embodiments include the probe assembly (e.g., the transducers that transmit and receive ultrasound waves), the data digitizer (e.g., the multiplexer 112, controller 114 and preprocessor 116) and the processing pipeline (e.g., processor 118 including machine learning model 120 and user interface 122) that receives ultrasound data and generates predictions as to the likelihood of a condition affecting the anatomical region of interest. The apparatus may be battery-operated or wired.

In some embodiments, the processor 118 is provided within a housing that includes the probe assembly 150, electrical interconnection layer 140 and processing assembly 110. In other embodiments, the processor 118 may be provided external to the probe assembly 150 and electrical interconnection layer 140 (and any housing that includes the probe assembly 150 and electrical interconnection layer 140), or even external to other elements of the processing assembly 110.

In some embodiments, there may be a connectivity module (not shown) that interfaces with one or more Electronic Medical Record (EMR) system, e.g., via a wireless interface, such as Bluetooth™ or Wi-Fi™.

Generally, the shape and form of the apparatus or the probe device embodying system 100 permits easy placement of the device on the body (e.g., torso) of a patient, and does not require precise positioning or troubleshooting to obtain suitable ultrasound data. In particular, the probe is adapted to be fixed in place over an anatomical region of interest.

To enhance acoustic coupling, a coupling layer 170 may be provided between the probe assembly 150 and the patient's body 180. In some embodiments, the coupling layer 170 may be a separate coupling gel pad. One example is the Aquaflex® Ultrasound Gel Pad sold by Parker Laboratories, Inc. In some cases, an adhesive suitable for use on skin may also be provided on the coupling gel pad, to assist in securing the transducer to the patient's body and help prevent unwanted movement. However, in other embodiments, a coupling gel may also be used as the coupling layer 170.

User interface 122 may include an output device for indicating the likelihood of the condition affecting the anatomical region of interest. In at least some embodiments, this user interface omits ultrasound images and instead presents only data representative of the likelihood of the condition, such as a numerical value representing the prediction confidence, a natural language output (e.g., “high”, “medium”, “low”), or a color-coded output (e.g., “green”, “yellow”, “red”). Accordingly, the output device may be an indicator light (e.g., light emitting diode (LED)), a seven-segment display, a dot matrix display, a liquid crystal display or any other graphic display. In some cases, where the system 100 or apparatus comprises a housing, the display may be provided on the housing. In some cases, the user interface may also be used to provide guidance to the user, such as suggesting alternative placement of the apparatus or the probe, or indicating that a prediction could not be generated based on the data or lack thereof.

Referring now to FIGS. 2A to 2E, there are illustrated example arrangements of transducers for a probe assembly. The transducer arrangement refers to the geometrical arrangement of transducers (and their associated transducer elements) within a single probe assembly, such as probe assembly 150.

In conventional clinical practice, a single array transducer is used to investigate multiple anatomical locations in sequence based on a certain clinical protocol.

However, in at least some of the described embodiments, multiple transducers may be provided in a geometrical arrangement that maximizes the likelihood of acquiring clinical significantly data without moving the probe assembly once fixed generally in place over an anatomical region of interest, and without using anatomical imaging or technician expertise to guide the placement and acquisition.

Depending on the type of transducer, the output of each transducer may be 1D (A-Mode), 2D (B-Mode) or 3D data sets as a function of time, and the system may generate one or multiple ultrasound beams and can control location, shape and direction of each beam.

In at least some of the described embodiments, all transducers are placed in the same assembly and housing within a predefined geometrical arrangement. The predefined geometrical arrangement may vary based on the anatomical region of interest or the patient's body size.

In some other embodiments, disposable or reusable patch-type transducers may be used, in which case transducer patches may be arranged according to the predefined geometrical arrangement.

A comparison of various example geometrical arrangements and the capabilities they enable is provided in Table A.

TABLE A
Transducer arrangements

| | Single | Multiple | Single | Multiple | |
|---|---|---|---|---|---|
| | element | element | 1D or 2D | 1D | Digital |
| | transducer | transducer | array | array | transducer |
| # of elements per | 1 | ~8 | 16-32 | 64-192 | >1000 |
| transducer | | | | | |
| Channels | 1 | ~8 | 16-32 | 32-64 | >1000 |
| M-Mode capability | y | y | y | y | y |
| Dynamic focusing | n | y | y | y | y |
| Beam steering | n | n | y | y | y |
| 2D imaging | n | n | n | y | y |
| 3D Imaging | n | n | n | n | y |
| Handheld | y | y | y | n | n |
| Portable/cart | y | y | y | y | y |

Referring now to FIG. 2A, there is illustrated a single element transducer arrangement 250a. In the arrangement shown, the single element transducer arrangement has N transducers arranged in a compact hexagonal array. In this example, N is 7, however, there may be fewer or more transducers and the arrangement need not be hexagonal. For example, the arrangement may be designed to cover an anatomical feature of interest, which may result in a shape that is oblong or irregular. In this simplest implementation, each transducer 260a is a single element that can generate only one single beam with a fixed shape and fixed direction. Together, the transducers 260a can generate N M-mode data sets.

Referring now to FIG. 2B, there is illustrated a multiple element transducer arrangement 250b. In the arrangement shown, the multiple element transducer arrangement has N transducers arranged in a compact hexagonal array. In this example, N is 7 but there may be fewer or more transducers and the arrangement need not be hexagonal. For example, the arrangement may be designed to cover an anatomical feature of interest, which may result in a shape that is oblong or irregular. Each transducer 260b has multiple transducer elements, enabling beam focusing to be performed.

Referring now to FIG. 20, there is illustrated an array transducer arrangement 250c. In the arrangement shown, the transducer arrangement has N×M transducers arranged in an array. In this example, N and M are each 6 but there may be fewer or more transducers and the arrangement need not be square. For example, the arrangement may be designed to cover an anatomical feature of interest, which may result in a longer width than height of the array. Each transducer 260c may be a single element or a multiple element transducer. In addition to beam focusing, arrangement 250c, 250c′ enables limited beam steering to be performed.

Referring now to FIG. 2D, there is illustrated an array-of-arrays transducer arrangement 250d. In the arrangement shown, the transducer arrangement has N 1D transducer arrays arranged in a compact arrangement surrounding a central array. In this example, N is 5, however, there may be fewer or more transducer arrays and the arrangement need not be rounded in shape. For example, the arrangement may be designed to cover an anatomical feature of interest, which may result in a longer width than height of the arrangement. Each transducer array 260d may have any number of individual transducers, which in turn may have any number of transducer elements. In addition to beam focusing and beam steering, arrangement 250d enables 2D imaging to be performed. The array-of-arrays transducer arrangement can be used to generate N 2D data sets.

Referring now to FIG. 2E, there is illustrated a digital transducer arrangement 250e. In the arrangement shown, the transducer arrangement has multiple digital transducers arranged in a compact arrangement. Each digital transducer 260e may be formed of an array of MEMS transducers. However, there may be fewer or more digital transducers and the arrangement need not be rounded in shape. For example, the arrangement may be designed to cover an anatomical feature of interest, which may result in a longer width than height of the arrangement. In addition to beam focusing and beam steering, arrangement 250e enables both 2D and 3D imaging to be performed.

Generally, the footprint of each transducer arrangement is large enough to cover the entire anatomical region of interest, but small enough to facilitate acoustic coupling of the entire surface. There may be different sizes of the same arrangement based on the anatomy of the patient (e.g., small sizes for children, larger sizes for adults). Different geometries may be used, e.g., square, rectangular, oval, etc.

Within an arrangement, there may be N transducers or, in some cases, an M×N rectangular array. As described elsewhere herein, subsets of the transducers may be used to obtain an included data set, and these transducers need not be contiguous in the arrangement.

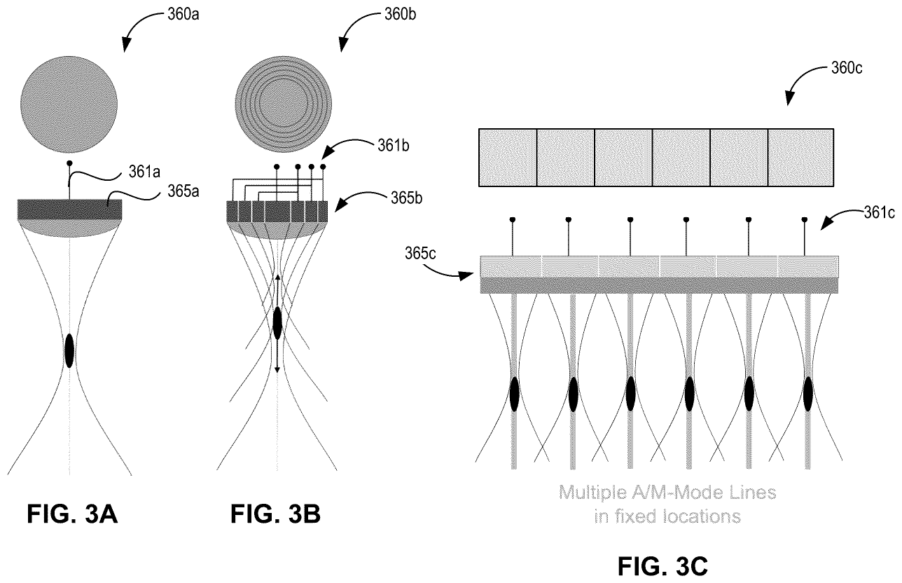

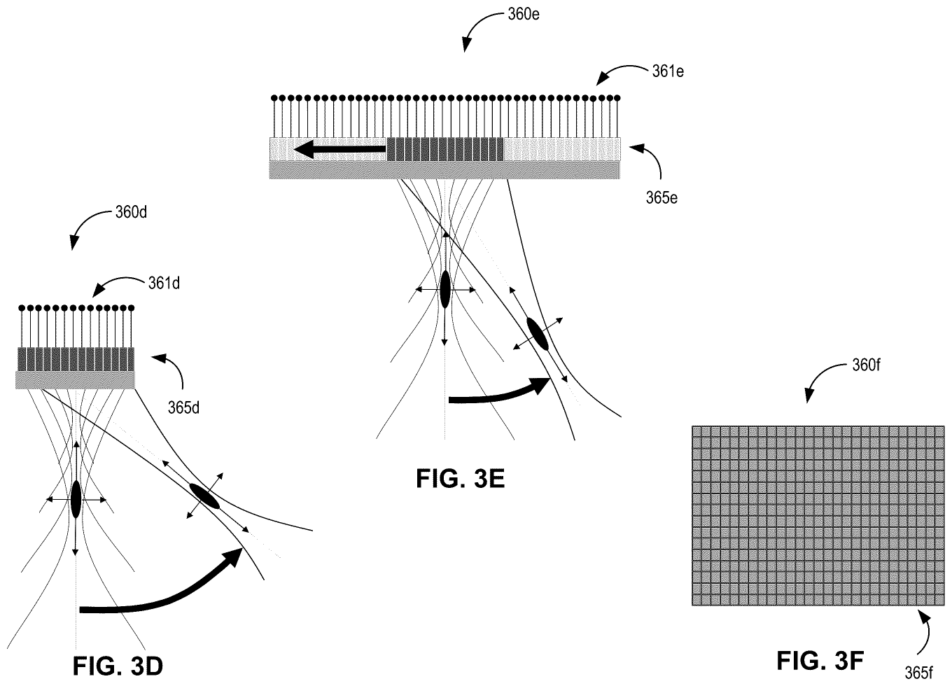

Referring now to FIGS. 3A to 3F, there are illustrated various types of transducers, both single element and multiple element.

FIG. 3A illustrates a single element transducer 360a comprising a single transducer element 365a, which is controlled by a control wire 361a. A single element transducer is simpler than other transducers but has a fixed focus and little or no ability to perform beam steering. Accordingly, it is difficult to produce 2D or 3D ultrasound data without manipulation of the transducer itself.

FIG. 3B illustrates a multiple element transducer 360b, in which each transducer element 365b is controlled by an individual control wire 361b. In this example, the transducer 360b is an annular array of transducer elements. The annular array transducer has concentric ring-shaped (i.e., annular) elements that can be individually controlled, enabling dynamic focusing in the axial dimension.

FIG. 3C illustrates a 1D array of single element transducers 360c, which has multiple individually controlled elements aligned in a single line. Each transducer element 365c is controlled by an individual control wire 361c. In this configuration, the array is a non-imaging array and the individual elements each have a fixed focus.

FIG. 3D illustrates a 1D array transducer with multiple elements 360d, commonly referred to as linear or convex array. Each transducer element 365d is controlled by an individual control wire 361d. This configuration allows for dynamic focusing but may have limited capabilities to perform beam steering.

FIG. 3E illustrates a 1D array transducer with multiple elements, 360e, commonly referred to as Phased Array. Each transducer element 365e is controlled by an individual control wire 361e. This configuration allows for electronic beam steering and dynamic focusing, which permits generating real-time high-resolution 2D images. By rapidly activating different groups of elements in sequence, the 1D phased array can steer the ultrasound beam across a wide field of view.

FIG. 3F illustrates a 2D array transducer 360f. A 2D array transducer, also known as a matrix array, consists of a grid of numerous tiny, independently controlled elements 365f arranged in both the vertical and horizontal dimensions. This design allows for electronic control over both the elevation and azimuthal planes, enabling real-time 3D (or 4D, with time as the fourth dimension) data acquisition. By manipulating the timing and intensity of the signals to each element, the 2D transducer can steer and focus the ultrasound beam in multiple directions without the need for mechanical movement, enhancing spatial resolution and image quality.

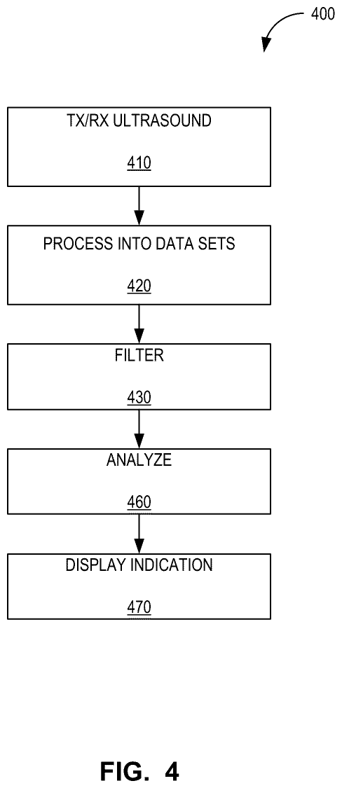

Referring now to FIG. 4, there is illustrated a flow chart diagram of an example method of detecting a likelihood of a condition affecting an anatomical region of interest. Method 400 may be carried out by, e.g., a system 100 or apparatus embodying system 100 and, in particular, by at least one processor of the system or apparatus.

Method 400 begins at 410 with the processor transmitting ultrasound waves via one or more transducers, and receiving reflected ultrasound waves (e.g., echoes). As described elsewhere, the processor may individually control each transducer. The processor may include a controller that generates and receives the signals, possibly via a multiplexer.

At 420, the processor, during an initialization phase, processes the data received from each transducer into one or more data sets. For example, the processor may determine which of the transducers' data is to form part of the included data set and/or the excluded data set. As described elsewhere herein, the processor may determine whether to include a transducer's data in the included data set by performing analysis of the data to identify, e.g., an artifact or other information that is not reflective of the anatomical region of interest.

At 430, the processor may perform filtering of the included data set. That is, even for data within the included data set, the processor may perform additional filtering. For example, the processor may perform low pass, high pass or bandpass filtering, or may truncate the data (e.g., crop boundary regions in 2-D data), and so forth. The preprocessing step generally serves to prepare the data for ingestion to a machine learning model to perform subsequent analysis.

At 460, the processor executes one or more machine learning model to identify the likelihood of a condition affecting the anatomical region of interest. The one or more machine learning model ingests data from the included subset as determined at 430 (or from data produced at 420, if no included subset is generated). If preprocessing has been performed, the ingested data is the preprocessed data. However, in some cases, the ingested data may be raw data directly received from the transducer (and multiplexer, controller and preprocessor, as the case may be). In some embodiments, an included subset is not generated, in which case the ingested data is all transducer data.

In some cases, the processor determines that the likelihood of a condition affecting the anatomical region of interest is met if the likelihood exceeds a predetermined threshold (which may be configurable).

At 470, the processor transmits an indication of the likelihood for display to a user, e.g., on a display. The processor may transmit the indication regardless of whether the likelihood exceeds the predetermined threshold, and the indication therefore provides information as to whether the likelihood exceeds the predetermined threshold or not. In some cases, the indication may include a numerical score or percentage corresponding to the likelihood.

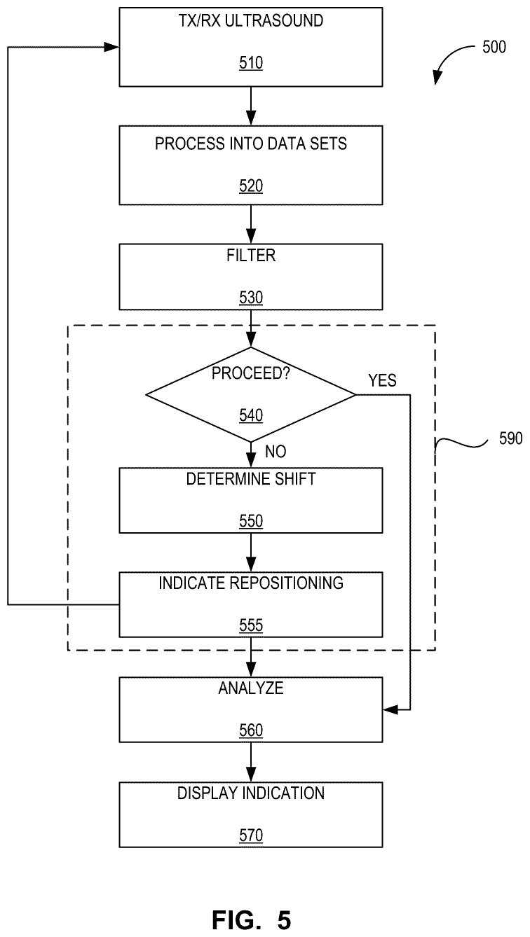

Referring now to FIG. 5, there is illustrated a flow chart diagram of another example method of detecting a likelihood of a condition affecting an anatomical region of interest. Method 500 may be carried out by, e.g., a system 100 or apparatus embodying system 100 and, in particular, by at least one processor of the system or apparatus. Method 500 is generally analogous to method 400, with the addition of a repositioning sub-process 590.

Method 500 begins at 510 with the processor transmitting ultrasound waves via one or more transducers, and receiving reflected ultrasound waves (e.g., echoes). As described elsewhere, the processor may individually control each transducer. The processor may include a controller that generates and receives the signals, possibly via a multiplexer.

At 520, the processor, during an initialization phase, processes the data received from each transducer into one or more data sets. For example, the processor may determine which of the transducers' data is to form part of the included data set and/or the excluded data set. As described elsewhere herein, the processor may determine whether to include a transducer's data in the included data set by performing analysis of the data to identify, e.g., an artifact or other information that is not reflective of the anatomical region of interest. Alternatively, the processor may determine whether to include transducer data by searching for markers in the data corresponding to the anatomical region of interest.

At 530, the processor may perform filtering of the included data set. That is, even for data within the included data set, the processor may perform additional filtering. For example, the processor may perform low pass, high pass or bandpass filtering, or may truncate the data (e.g., crop boundary regions in 2-D data), and so forth. The preprocessing step generally serves to prepare the data for ingestion to a machine learning model to perform subsequent analysis.

At 540, a determination is made by the processor whether to proceed with further analysis, as part of a repositioning sub-process 590. The determination may be performed based on the quality of the data in the included subset, or the lack of sufficient data in the included subset, or on the quality of the data overall. For example, if some or all of the available data is excluded from the included subset (e.g., the number of excluded transducers exceeds an exclusion threshold), either because the data contains artifacts or lacks markers, then the processor may determine that the apparatus or probe assembly is not positioned properly.

Optionally, at 550, the processor may analyze the available data (e.g., known markers), e.g., using a machine learning model, and determine an offset by which the apparatus or probe assembly should be repositioned to improve the quality of the scan and the resultant data.

At 555, the processor generates and displays, e.g., on a display, an indication that the apparatus or probe assembly should be repositioned. If the processor has determined an offset at 550, an indication of the offset may be displayed also. For example, the indication may contain an instruction to reposition the device “down by 2 cm” or “up by 3 cm” and so forth. Once the apparatus or probe assembly is repositioned (or after a delay), the processor returns to 510. Otherwise, if no repositioning was determined to be necessary at 540, the processor proceeds to 560.

At 560, the processor executes one or more machine learning model to identify the likelihood of a condition affecting the anatomical region of interest. The one or more machine learning model ingests data from the included subset as determined at 530 (or from data produced at 520, if no included subset is generated). If preprocessing has been performed, the ingested data is the preprocessed data. However, in some cases, the ingested data may be raw data directly received from the transducer (and multiplexer, controller and preprocessor, as the case may be). In some embodiments, an included subset is not generated, in which case the ingested data is all transducer data.

In some cases, the processor determines that the likelihood of a condition affecting the anatomical region of interest is met if the likelihood exceeds a predetermined threshold (which may be configurable).

At 570, the processor transmits an indication of the likelihood for display to a user, e.g., on a display. The processor may transmit the indication regardless of whether the likelihood exceeds the predetermined threshold, and the indication therefore provides information as to whether the likelihood exceeds the predetermined threshold or not. In some cases, the indication may include a numerical score or percentage corresponding to the likelihood.

As described elsewhere herein, the described embodiments may execute a machine learning model to make predictions regarding the likelihood of a condition that affects an anatomical region of interest. One example of such a condition is pneumothorax (air in the pleural space). In ultrasound, pneumothorax can be detected through the presence of “lung sliding,” a characteristic motion observed when the ultrasound probe is placed on the chest wall over the lungs. When the lungs are healthy and properly inflated, they move against the chest wall during respiration. This movement creates a dynamic interface between the parietal pleura (lining the chest wall) and the visceral pleura (covering the lungs). When observed on ultrasound, this movement appears as a shimmering or sliding motion of the pleural line, hence the term “lung sliding.”

Lung sliding is a reassuring sign of lung health and proper lung expansion. It is typically absent or diminished in conditions where there is air or fluid between the pleural layers, such as pneumothorax or pleural effusion (fluid in the pleural space). Thus, the presence or absence of lung sliding is an important diagnostic indicator in assessing pulmonary conditions using ultrasound.

In at least one embodiment, a machine learning model may ingest lung ultrasound data (e.g., M-mode or B-mode) as input and return a binary prediction for whether there is evidence of absent lung sliding.

Generally, if absent lung sliding is present anywhere throughout the data in the spatial or temporal dimensions, the system's decision is “Absent Lung Sliding” (even if some regions show evidence of lung sliding). Conversely, if lung sliding is present everywhere throughout the data, the system's decision is “Lung Sliding”, meaning that the system can assist in ruling out a diagnosis of pneumothorax (PTX) at the site of the ultrasound probe.

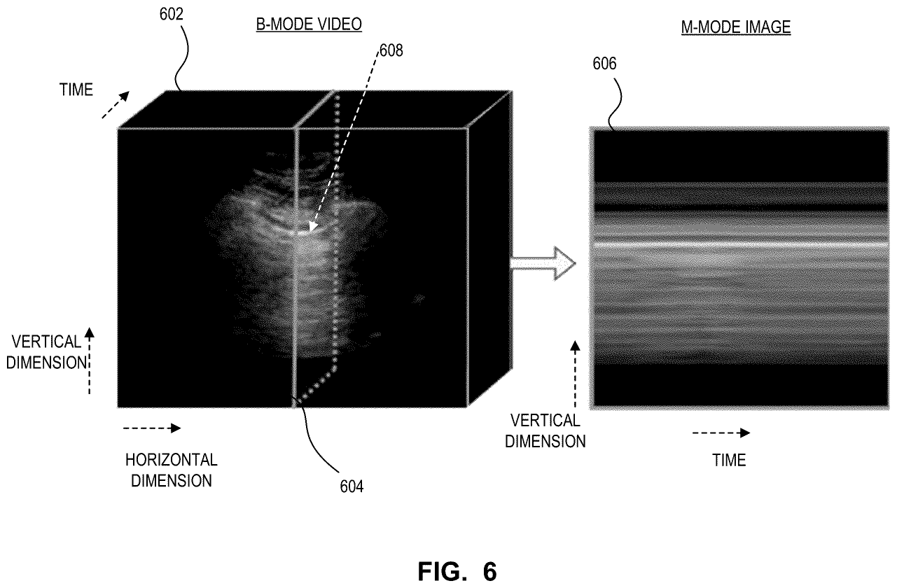

In this example, the machine learning model can make use of M-mode ultrasound data, which can be described as a vertical slice of B-mode data through time. As illustrated in FIG. 6, B-mode video 602 is illustrated as a series of video frames each having a vertical dimension and a horizontal dimension. The video frames may vary over time. A vertical slice 604 through each of the B-mode video frames is used to form an M-mode image 606. The horizontal dimension of M-mode data is time, and the vertical dimension is the vertical dimension of the data. In some cases, M-mode imaging refers to motion mode imaging. M-mode imaging includes axial and temporal resolution of structures, in which a single scan line may be emitted, received, and displayed graphically. In some cases, the B-mode video 602 captures a pleural line 608. In some cases, the computing system and method described herein generates one or more M-mode images that intersect the pleural line 608 and processes the same to determine that lung sliding is present or absent.

The machine learning model may use a deep convolutional neural network to predict whether M-mode data contains evidence for lung sliding or absent lung sliding. M-mode data is eligible for consideration if it intersects the pleural line artifact.

In at least some embodiments, the described methods are split into three broad modules:

Pleural line detection & M-mode designation: This module outputs a bounding box that contains the pleural line artifact throughout the duration of the data. The box can be described as the location of the top left corner, along with its width and height. All M-mode data that intersects the pleural line bounding boxes is eligible for classifier prediction.

Classification of M-modes: Each instance or frame of M-mode data is passed through a convolutional neural network binary classifier that predicts lung sliding (negative class) or absent lung sliding (positive class). It outputs confidence p in range [0, 1]. The predicted class is negative if p is less than classification threshold t, or positive otherwise.

Clip Prediction Algorithm: The series of constituent M-mode-level prediction confidences for each B-mode are translated into a binary prediction for the entire clip, indicating whether the clip contains evidence of absent lung sliding.

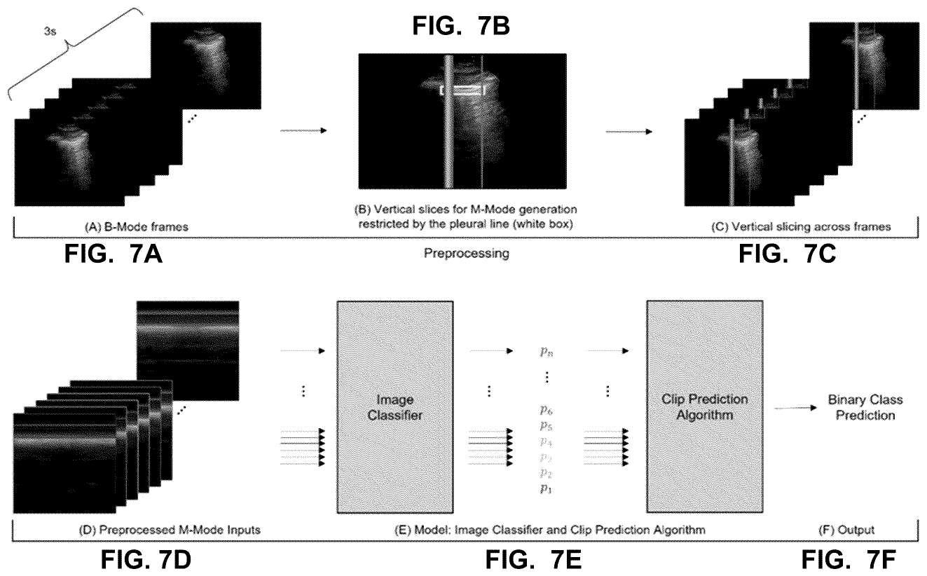

An overview of the methodology is illustrated in FIGS. 7A to 7F, which provide a schematic representation of the methods underlying each 3-second clip segment prediction. Referring to FIGS. 7A to 7D, for each masked 3-second B-Mode data (clip), a set of n M-Mode frames are generated during preprocessing by slicing through the pleural line at n different positions through time. n is an integer greater than or equal to 1. In particular, FIG. 7A shows a set of B-mode frames (in a time series) that form the 3-second video clip. FIG. 7B shows n vertical slices demarcated in a given B-mode frames that are used for M-mode image generation. In some cases, the vertical slices for M-mode image generation are bound to a region of interest, such as the pleural line shown by the box in FIG. 7B. FIG. 7C shows vertical slicing across each frame in the set of B-mode frames. In some cases, the operations and data components shown in FIGS. 7A to 7D are executed or implemented by the processor 118 of FIG. 1.

In FIG. 7E, these n inputs are sent to the model, which consists of a classifier (e.g., image classifier) and a clip prediction algorithm. The image classifier predicts the confidence p of absent lung sliding at the M-Mode-level. The latter converts the resulting series of n M-Mode-level predictions into a single binary prediction (shown in FIG. 7F) of “Lung Sliding Absent” or “Lung Sliding Present” for the clip segment. In some cases, the image classifier is a convolutional neural network binary classifier. In some cases, the operations and data components shown in FIGS. 7E and 7F are executed, or implemented, by the processor 118 of FIG. 1.

The use of a neural network for detecting absent lung sliding in humans based on M-mode lung ultrasound images has been suggested by Jaščur et al., “Detecting the Absence of Lung Sliding in Lung Ultrasounds Using Deep Learning,” Appl. Sci. 2021, 11, 6976, https://doi.org/10.3390/app11156,976 and VanBerlo et al., “Accurate assessment of the lung sliding artefact on lung ultrasonography using a deep learning approach,” Comput Biol Med. 2022 September; 148:105953, doi: 10.1016/j.compbiomed.2022.105953, Epub 2022 Aug. 9, PMID: 35985186.

In some cases, the described embodiments provided herein improve on prior approaches in the following areas:
Pleural line detection—multiple strategies could be used for identifying the pleural line. The described embodiments employ two approaches to this: (1) a machine learning object detection approach and (2) a novel explicit algorithmic approach that relies on computer vision methods.
Clip classification methods—an important special case of the positive class occurs in lung point scenarios. In lung point, an artifact that is definitive of PTX, part of the pleural line exhibits lung sliding, and the other part exhibits absent lung sliding. The most clinically significant finding is the existence of absent lung sliding; therefore, the Clip Prediction Algorithm would output “Absent Lung Sliding” (or some other indication of absence of lung sliding) under these circumstances. The described embodiments provide multiple approaches for distilling multiple M-mode-level confidence levels into a single binary decision for whether a B-mode clip contains evidence of absent lung sliding.

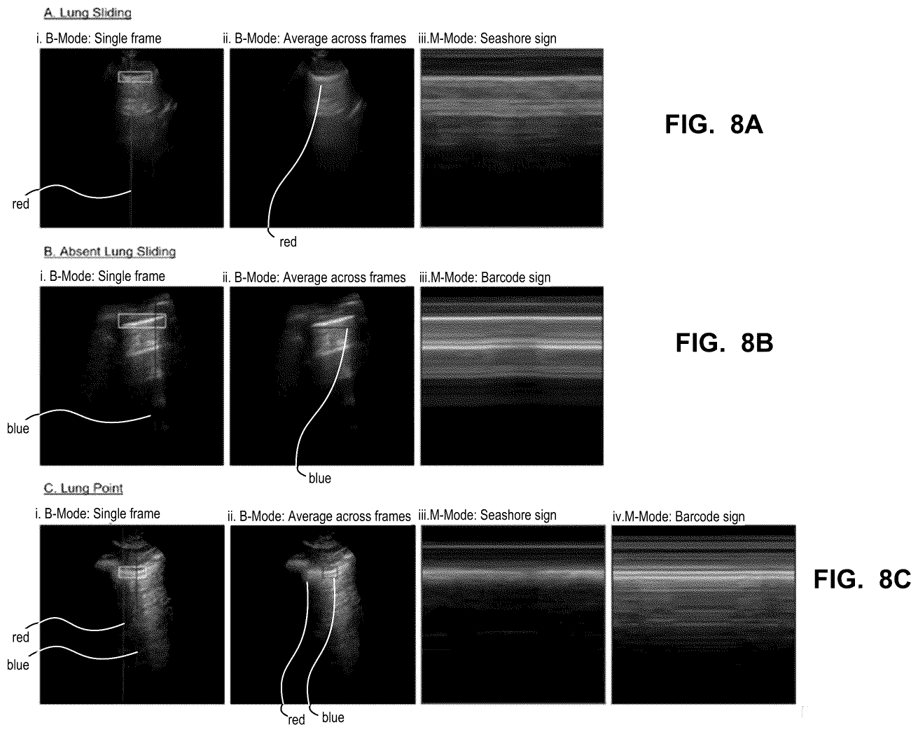

FIGS. 8A to 8C provide a clear visual representation of the lung point scenario and how it compares to the more common scenarios where there is only evidence of either present or absent lung sliding.

In FIGS. 8A to 8C, there is shown a comparison of the lung sliding (FIG. 8A), absent lung sliding (FIG. 8B), and lung point (FIG. 8C) artifacts on B-Mode (panels i and ii) and M-Mode (panels iii and iv) ultrasound. The color red in the vertical lines and/or bounding boxes signifies the presence of lung sliding. The color blue in the vertical lines and/or bounding boxes signifies the absence of lung sliding. Bounding boxes highlight the location of the pleural line on single and averaged B-Mode frames in panels i and ii, respectively. Vertical lines indicate the B-Mode slices used to produce the M-Mode images displayed in panels iii and iv. In FIG. 8A: The lung sliding artifact is present across the entirety of the pleural line. M-Modes sliced through any horizontal index intersecting the pleural line will display the seashore sign, for example as described in Lichtenstein, “Whole Body Ultrasonography in the Critically Ill,” Springer Science & Business Media (2010), doi: https://doi.org/10.1007/978-3-642-05328-3. In FIG. 8B: The lung sliding artifact is absent across the entirety of the pleural line. M-Modes sliced through any horizontal index intersecting the pleural line will display the barcode sign. In FIG. 8C: A transition from a sliding to a static pleural line is visualized (i.e., the lung sliding artifact is both present and absent in the same B-Mode). M-Modes sliced at indices to the left of the lung point will display the seashore sign. M-Modes sliced at indices to the right of the lung point will display the barcode sign.

### Pleural Line Detection & M-Mode Designation

In this section of the described approach, eligible M-mode images are extracted from input B-mode ultrasound data. FIGS. 7A to 7D summarize the product of this step. Eligible M-mode data includes frames that intersect the pleural line. It is therefore helpful to identify the horizontal bounds of the pleural line. The described embodiments provide two methods for determining the location of the pleural line. Both methods output possible x-coordinates that intersect the pleural line, permitting M-mode extraction.

When the computing system has identified the M-mode bounding box(es) (e.g., in some cases using user input or in some cases automatically (, the B-Mode clip is divided into standardized segments, known as “clip segments”, that are each 3 seconds in duration (though other durations may also be used), consistent with the amount of time required for clinicians to interpret for lung sliding, for example as described in Lichtenstein, “Lung ultrasound in the critically ill,” Annals of intensive care, 4(1), 1-12 (2014). Clip segments are taken from the beginning of each clip, and segment overlap is permitted to ensure complete coverage. For example, if the clip is 7 seconds in duration, three clip segments will be produced; one for 0:00-0:03, one for 0:03-0:06, and one for 0:04-0:07. M-modes are produced for each x-coordinate within the pleural line bounding box(es), for each clip segment.

In some cases, different approaches are used for pleural line detection, which are described below.

In a first object detection approach, a machine learning model is trained to predict the locations and sizes of bounding boxes that may contain the pleural line. In some cases, lung ultrasound experts annotate several B-mode videos with frame-level bounding boxes. The boxes may be specified in either of the following manners:
(x, y) coordinate of top left corner, width, and height.
(x, y) coordinates of both the top left and bottom right corners

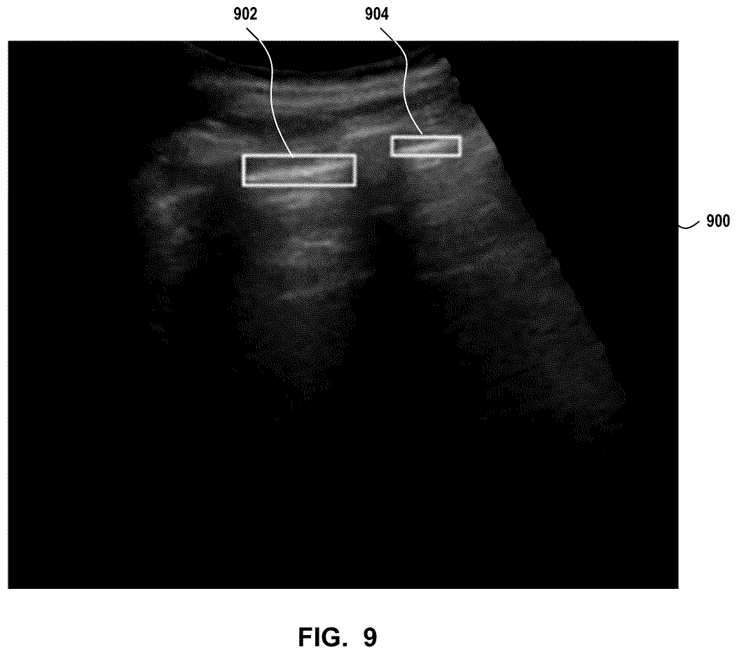

An object detection model with a standard architecture and objective function may be trained to output the location of one or more fragments of the pleural line. FIG. 9 provides an example of a B-mode image 900 where multiple pleural line fragments are separated by rib shadows. In some cases, detection architectures for image processing include, but are not limited to, the Ultralytics YOLO™, region-based convolutional neural network (RCNN) family, or single shot detector (SSD) families. Predicted bounding boxes 902 and 904 are shown around the fragments of a pleural line. Box predictions may be retained if their predicted confidence is above a threshold. Any x-coordinate within any of the predicted bounding boxes are valid locations at which an M-mode image may be taken.

In a second, explicitly programmed approach, and to avoid including a separate machine learning model in this method, the following procedure may be used to identify the single strongest pleural line fragment candidate. The values of the parameters are tuned for B-modes. The approach may be applied to either entire clips or to 3-second clip segments. In this example, ultrasound images and video data are used, although in other approaches it need not be converted into images or video.
1. Clip (segment) average: The pixel intensities are averaged across the time dimension of the B-mode video (see FIG. 8A panel ii, FIG. 8B panel ii, and FIG. 8C. panel ii for examples)
2. Normalization: Rescale the clip average's pixel values such that all pixel intensities are in [0, 1].
3. Increase contrast: Multiply each pixel x by e^{k*x}, where k is set to be 4.5 for best results. This increases the contrast of the image. Renormalize by dividing by the new maximum.
4. Radon Transform: Apply the Radon Transform (see, e.g., Roulston et al., “Synthesizing radar maps of polar regions with a Doppler-only method,” Applied Optics, 36(17), 3912-3919) for a range of angles in 70-110 degrees. Find the brightest point on the resulting sinogram. Use the angle of this point to rotate the image such that the pleural line, A-lines, and ribs are horizontally oriented.
5. Thresholding: Apply global thresholding with a lower bound of 40 to create a mask of where the pleural line might be. Apply adaptive thresholding with a block size of 21 and per-block mean subtraction constant of 5. Use the global thresholding mask to extract the region of interest from the adaptive threshold result.
6. Horizontal Erosion: Let w be the width of the image. Create a structuring element with shape (w//25, 1). Erode the image for 1 iteration. If this results in an image is black, restore the result of step 5.
7. Horizontal Dilation: Dilate the image with same structuring element from step 6 for 1 iteration.
8. Find contours: Apply a contour finding algorithm to retrieve the boundaries of each possible object in the image, which should include the pleural line.
9. Narrow down choices of contours: Let w and h be the width and height of the image, respectively. Eliminate contours with 0 area. Eliminate contours with an area that is less than equal to 3% of the image area and whose height is greater than or equal to a tenth of h or whose width is less than or equal to a twelfth of w. Eliminate contours with a perimeter-to-area ratio that is less than or equal to 0.3.
10. Choose best contour: Select the contour for which the sum of pixel intensities in coordinates that are below it (within x-coordinate bounds) is the greatest. If there are no contours left over from step 9, select the eliminated contour with the greatest width.
11. Double-check best contour: Identify all contours directly above or below the best contour. Among the current best contour and the newly identified contours, return the contour whose 20 brightest constituent pixel intensities have the greatest sum.

A description of a deep learning approach to M-mode classification for the absence or presence of lung sliding follows.

All M-modes images are resized to a fixed dimension and pixel intensities are rescaled to a fixed range. A convolutional neural network image binary classifier is trained to distinguish between present versus absent lung sliding. The output of the network is the confidence in absent lung sliding (p). Examples of lung point are excluded from the training and validation sets for the classifier so that a clip-wise label can be adopted, ensuring that all valid M-modes in the clip have the same label.

The output of this step is a prediction confidence for each M-mode in each 3-second clip segment. In some cases, the video clip is divided into m-second clip segments. In some cases, including the examples herein, m is 3 seconds. Other time lengths could be used to divide the video clip into segments.

A clip classification algorithm, which may be executed by the processor 118 of FIG. 1, receives the prediction confidences from each M-mode in each 3-second clip segment as input, and it outputs a binary decision for “Lung Sliding Present” or “Lung Sliding Absent” for the entire clip. Generally, the algorithm will output “Absent Lung Sliding” if there is any evidence of absent lung sliding at any point of the pleural line, throughout the duration of the clip. Since it is expected that there may be noise in the M-mode confidences, multiple methods may be used for applying this clinical logic. Alternative methods are described in the subsections below.

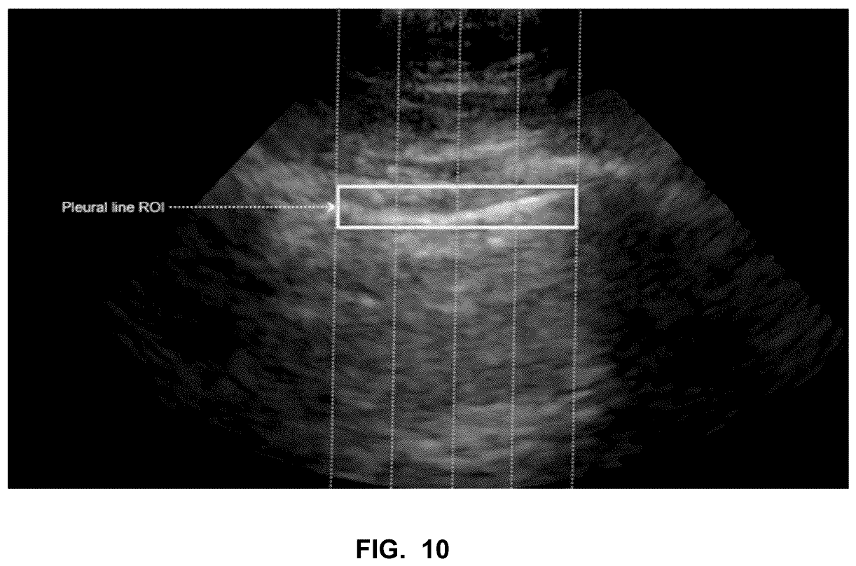

In some cases, the below definitions regarding clip segments are used throughout these methods:
Binning: Divide the pleural line bounding box into b horizontally spaced segments with equal width (see FIG. 10 for example)
Brightness: Sum of pixel intensities of an M-mode image.
Threshold: Classification threshold t. If prediction confidence p>t, then the prediction for an individual M-mode is “Absent Lung Sliding”. If p<t, the prediction is “Lung Sliding”.
Moving average: Given a list of M-mode prediction confidences in the same clip segment ordered from left to right, obtain a smoothed set of prediction confidences by taking the moving average with window size w, where w is an integer greater than 1.

In some cases, the methods involve the following:
1. For each 3-second clip segment:
   a. Perform binning for each clip segment, to divide the x-coordinates of the pleural line into contiguous chunks with equal width.
   b. Obtain a predicted class for each 3-second clip segment. Optionally preceded by taking a moving average of the M-mode prediction confidences for each clip segment. Clip segment predictions are obtained by checking each bin for absent lung sliding.
2. If any 3-second clip segment's prediction is “Absent Lung Sliding”, then the entire clip's prediction is “Absent Lung Sliding”.

Several embodiments are described, each of which are generally similar to each other, with the exception of step 1. Below are the descriptions of four embodiments. FIG. 10 provides a visual accompaniment to understanding the process of producing a prediction for a 3-second clip segment, given its M-mode prediction confidences.

### Method 1

1. For each 3-second clip segment:
   a. Perform binning for each clip segment, to divide the x-coordinates of the pleural line into contiguous chunks with equal width.
   b. Take the brightest M-mode image in each bin.
   c. Apply the classification threshold to the prediction confidence for each selected M-mode to get a class prediction for each bin.
   d. If any of the predictions are “Absent Lung Sliding”, the clip segment's prediction is set to “Absent Lung Sliding”.
2. If any clip segment's prediction is “Absent Lung Sliding”, then the entire clip's prediction is “Absent Lung Sliding”.

### Method 2

1. For each 3-second clip segment:
   a. Perform binning for each clip segment, to divide the x-coordinates of the pleural line into contiguous chunks with equal width.
   b. For each bin, determine the mean prediction confidence for its constituent M-modes.
   c. Apply the classification threshold to the averaged prediction confidence to get a class prediction for each bin.
   d. If any of the predictions are “Absent Lung Sliding”, the clip segment's prediction is set to “Absent Lung Sliding”.
2. If any clip segment's prediction is “Absent Lung Sliding”, then the entire clip's prediction is “Absent Lung Sliding”.

### Method 3

1. For each 3-second clip segment:
   a. Perform binning for each clip segment, to divide the x-coordinates of the pleural line into contiguous chunks with equal width.
   b. Replace the list of prediction confidences for the current clip segment with its moving average.
   c. Compute a moving average (with the same window size as in b.) of the brightness of each M-mode at each x-coordinate of the pleural line.
   d. Take the M-mode image in each bin with the greatest brightness moving average.
   e. Apply the classification threshold to the prediction confidence for each selected M-mode to get a class prediction for each bin.
   f. If any of the predictions are “Absent Lung Sliding”, the clip segment's prediction is set to “Absent Lung Sliding”.
2. If any clip segment's prediction is “Absent Lung Sliding”, then the entire clip's prediction is “Absent Lung Sliding”.

### Method 4

1. For each 3-second clip segment:
   a. Perform binning for each clip segment, to divide the x-coordinates of the pleural line into contiguous chunks with equal width.
   b. Replace the list of prediction confidences for the current clip segment with its moving average.
   c. Take the M-mode corresponding to the midpoint of each bin.
   d. Apply the classification threshold to the prediction confidence for each selected M-mode to get a class prediction for each bin.
   e. If any of the predictions are “Absent Lung Sliding”, the clip segment's prediction is set to “Absent Lung Sliding”.
2. If any clip segment's prediction is “Absent Lung Sliding”, then the entire clip's prediction is “Absent Lung Sliding”.

### Method 5

1. For each 3-second clip segment:
   a. Perform binning for each clip segment, to divide the x-coordinates of the pleural line into contiguous chunks with equal width.
   b. In each bin, take the M-mode corresponding to the midpoint of the range of prediction confidences for that bin.
   c. Apply the classification threshold to the prediction confidence for each selected M-mode to get a class prediction for each bin.
   d. If any of the predictions are “Absent Lung Sliding”, the clip segment's prediction is set to “Absent Lung Sliding”.
2. If any clip segment's prediction is “Absent Lung Sliding”, then the entire clip's prediction is “Absent Lung Sliding”.

### Method 6

1. For each 3-second clip segment:
   a. Perform binning for each clip segment, to divide the x-coordinates of the pleural line into contiguous chunks with equal width.
   b. In each bin, take the M-mode corresponding to the median of the prediction confidences for that bin.
   c. Apply the classification threshold to the prediction confidence for each selected M-mode to get a class prediction for each bin.
   d. If any of the predictions are “Absent Lung Sliding”, the clip segment's prediction is set to “Absent Lung Sliding”.
2. If any clip segment's prediction is “Absent Lung Sliding”, then the entire clip's prediction is “Absent Lung Sliding”.

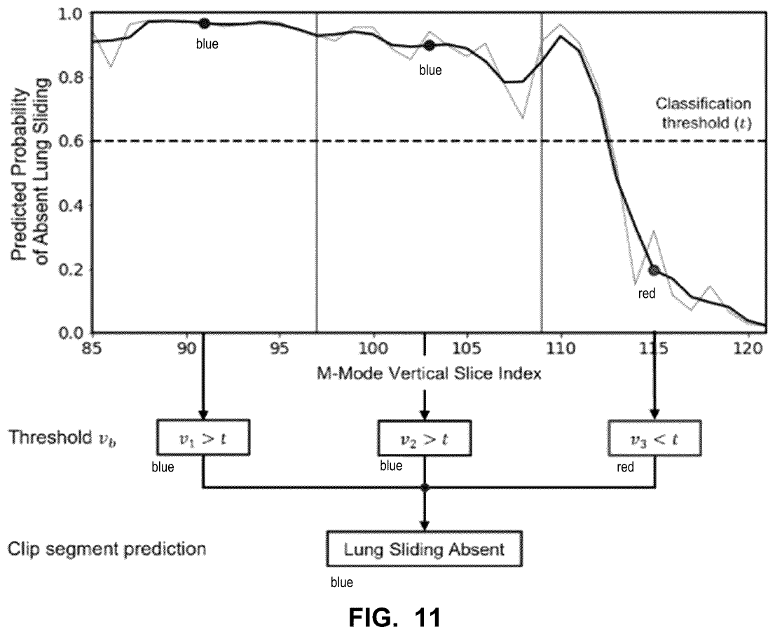

FIG. 11 provides a visual representation of step 1 for Method 4 applied to a single clip segment. For a given 3-second clip segment, the contiguous set of M-Mode-level prediction probabilities outputted by the image classifier (grey curve) are smoothed by computing a moving average with window size w (bolded black curve). The smoothed probabilities are divided into b bins and those at the midpoint of each bin (v_{b}; circular markers) are selected. If any of the selected values exceed the classification threshold (t; dashed line), then a label of “Lung Sliding Absent” (blue) is assigned to that clip segment. Otherwise, a label of “Lung Sliding Present” (red) is assigned. In this example, w=3, b=3 and t=0.6.

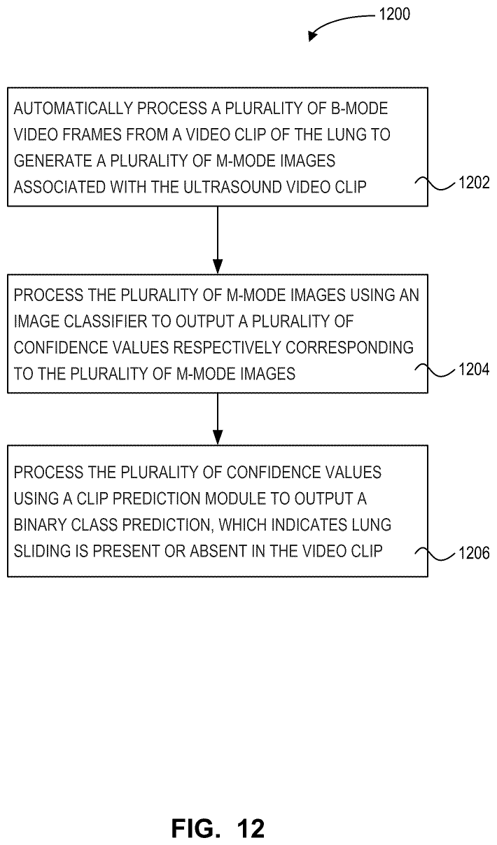

Referring to FIG. 12, in some cases, a process 1200 executed by the processor 118 of FIG. 1 includes the following operations.
Block 1202: Automatically processing a plurality of B-mode video frames from a video clip of the lung to generate a plurality of M-mode images associated with the ultrasound video clip.
Block 1204: Processing the plurality of M-mode images using an image classifier to output a plurality of confidence values respectively corresponding to the plurality of M-mode images; and
Block 1206: Processing the plurality of confidence values using a clip prediction module to output a binary class prediction, which indicates lung sliding is present or absent in the video clip.

The machine learning models described with reference to FIGS. 6 to 12 are only example machine learning models, and other machine learning approaches may be used together with the systems, methods and apparatus described herein and with particular reference to FIGS. 1 to 5.

For example, the machine learning models may be trained to identify B-lines and/or pleural effusion. In such case, the processor may further process image-wise B-mode predictions into a single video prediction using multiple contiguous images to eliminate prediction noise.

Furthermore, to guard against unwanted movement of the probe assembly while using pleural line detection, the machine learning models may include additional object detection models. For example, the machine learning models may identify, combine and track bounding box predictions for the pleural line. In some cases, the same machine learning model may detect the presence of B-lines and localize the pleural line.

Still further, the machine learning models may include a network in which a base model extracts features that are fed to multiple, smaller lightweight classifiers, each specific to certain types of artifacts.

Various systems or processes have been described to provide examples of embodiments of the claimed subject matter. No such example embodiment described limits any claim and any claim may cover processes or systems that differ from those described. The claims are not limited to systems or processes having all the features of any one system or process described above or to features common to multiple or all the systems or processes described above. It is possible that a system or process described above is not an embodiment of any exclusive right granted by issuance of this patent application. Any subject matter described above and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth to provide a thorough understanding of the subject matter described herein. However, it will be understood by those of ordinary skill in the art that the subject matter described herein may be practiced without these specific details. In other instances, well-known methods, procedures, and components have not been described in detail so as not to obscure the subject matter described herein.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

Terms of degree such as “substantially”, “about”, and “approximately” as used herein mean a reasonable amount of deviation of the modified term such that the result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the result is not significantly changed.

Some elements herein may be identified by a part number, which is composed of a base number followed by an alphabetical or numerical suffix (e.g. 112a, or 112-1). All elements with a common base number may be referred to collectively or generically using the base number without a suffix (e.g. 112). Similarly, analogous elements may have reference characters with the same two least significant digits (e.g., transducers 260 or 360 are analogous to transducers 160).

The systems and methods described herein may be implemented as a combination of hardware or software. In some cases, the systems and methods described herein may be implemented, at least in part, by using one or more computer programs, executing on one or more programmable devices including at least one processing element, and a data storage element (including volatile and non-volatile memory and/or storage elements). These systems may also have at least one input device (e.g. a pushbutton keyboard, mouse, a touchscreen, and the like), and at least one output device (e.g. a display screen, a printer, a wireless radio, and the like) depending on the nature of the device. Further, in some examples, one or more of the systems and methods described herein may be implemented in or as part of a distributed or cloud-based computing system having multiple computing components distributed across a computing network.

Some elements that are used to implement at least part of the systems, methods, and apparatuses described herein may be implemented via software that is written in a high-level procedural language such as object-oriented programming language. Accordingly, the program code may be written in any suitable programming language such as Python or Java, for example. Alternatively, or in addition thereto, some of these elements implemented via software may be written in assembly language, machine language or firmware as needed. In either case, the language may be a compiled or interpreted language.

At least some of these software programs may be stored on a storage media (e.g., a computer readable medium such as, but not limited to, read-only memory, magnetic disk, optical disc) or a device that is readable by a general or special purpose programmable device. The software program code, when read by the programmable device, configures the programmable device to operate in a new, specific, and predefined manner to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. The computer usable instructions may also be in various formats, including compiled and non-compiled code.

While the above description provides examples of one or more processes or systems, it will be appreciated that other processes or systems may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

1. An ultrasound apparatus for scanning an anatomical region of interest, the apparatus comprising:
a housing adapted to be fixed in place over the anatomical region of interest;
a plurality of transducers provided in the housing and positioned in a geometrical arrangement for scanning the anatomical region of interest while the housing is fixed in place over the anatomical region of interest, the plurality of transducers configured to emit a plurality of ultrasound waves and receive a plurality of echoes produced by the plurality of ultrasound waves; and
at least one processor configured to:
categorize the plurality of transducers into one or more included transducers and one or more excluded transducers based on the plurality of echoes, wherein the one or more included transducers provide relevant data for identifying a likelihood of a condition affecting the anatomical region of interest;
generate an included dataset that includes only echoes of the plurality of echoes from the one or more included transducers; and
process the included dataset using a machine learning model to identify the likelihood of the condition affecting the anatomical region of interest.

2. The apparatus of claim 1, wherein each of the plurality of transducers comprises a single element with fixed focus.

3. The apparatus of claim 1, wherein each of the plurality of transducers comprises one or more annular element.

4. The apparatus of claim 1, wherein each of the plurality of transducers comprises a 1-D array of elements.

5. The apparatus of claim 1, wherein each of the plurality of transducers comprises a 2-D array of elements.

6. The apparatus of claim 1, wherein the geometrical arrangement covers an area larger than the anatomical region of interest.

7. The apparatus of claim 1, wherein the anatomical region of interest has an area between 25 cm2 and 400 cm2.

8. The apparatus of claim 1, wherein the anatomical region of interest is selected from the group consisting of trachea, bronchi, bronchioles, alveoli, pleurae and pleural cavity.

9. The apparatus of claim 1, wherein the geometrical arrangement is a 1-D geometrical arrangement.

10. The apparatus of claim 1, wherein the geometrical arrangement is a 2-D geometrical arrangement.

11. The apparatus of claim 1, wherein the housing is flexible.

12. The apparatus of claim 1, further comprising a stick-to-skin adhesive provided on the housing for fixing the housing in place over the anatomical region of interest.

13. The apparatus of claim 1, further comprising a coupling gel provided on the housing for acoustically coupling the plurality of transducers.

14. The apparatus of claim 1, wherein the at least one processor is configured to individually control the plurality of transducers.

15-16. (canceled)

17. The apparatus of claim 1, further comprising a display provided on the housing, wherein, when the likelihood exceeds a threshold percentage, the at least one processor transmits an indication to the display.

18. The apparatus of claim 14, wherein the at least one processor is provided within the housing.

19. The apparatus of claim 14, wherein the at least one processor is provided external to the housing.

20. The apparatus of claim 1, wherein each of the plurality of transducers comprises at least one element, and the elements are selected from the group consisting of: crystal, ceramic with piezoelectric properties, and MEMS.
